(19) United States
(12) Patent Application Publication
(10) Pub. No.: US 2009/0137894 A1
(43) Pub. Date: May 28, 2009

(54) Breast measurement device and bra fitting system

(76) Inventor: Les Olson, Issaquah, WA (US)

Correspondence Address: DEAN A. CRAINE, SUITE 140, 400 - 112TH AVENUE NE, BELLEVUE, WA, 98004, US

(21) Appl. No.: 12/221,954

(22) Filed: Aug. 8, 2008

Related U.S. Application Data
(60) Provisional application No. 61/004,381, filed on Nov. 26, 2007.

(51) Int. Cl. A61B 5/05; A61B 5/103

(52) U.S. Cl. 600/407; 600/587

(57) ABSTRACT

A breast measurement device and method of accurately measuring a bra for a woman by submerging the woman's upper torso in a body of water that allows the buoyancy of each breast to lift and re-conform the breasts to their natural shape. A scanning machine is then used to measure the woman's upper torso and the submerged, lifted breasts to obtain their exact size, volume and location which then can be used to select a properly fitting bra which holds and supports the breasts in their natural position. In the preferred embodiment, the system will include a transparent tank filled with water in which a woman sits upright. A scanning machine then moves around tank and aimed at the woman's upper torso and the breasts. A digital file is then created and stored that is then used to select a proper fitting bra.

This is a utility patent application which claims benefit of U.S. Provisional Application No. 61/004,381 filed on Nov. 26, 2007.

### BACKGROUND OF THE INVENTION

1. Field of the Invention

This invention pertains to a method of measurement for measuring breasts and for fitting of bras.

2. Description of the Related Art

The term ‘bra’ is conveniently used to encompass all clothing designed to closely fit a wearer's breast, thus including other forms of lingerie such as body suits, swimwear, and other sportswear such as leotards.

Bras are important components of a woman's lingerie wardrobe that makes the woman feel comfortable and look beautiful. Because bras are available in many types of fabrics, style, and sizes and because breast tissue can change do to pregnancy, exercising, the use of birth control pills, large weight fluctuations and age, a woman's bra size often changes during her lifetime. In some instances, one breast may be one size and the other breast may be another size.

As women age, hormonal changes, redistribution of fat, and the continuous gravitational pull on the breasts causes changes in the woman's upper torso and breast sagging. In some instances, the breasts become asymmetrical.

Unfortunately, current bra fitting techniques do not adequately measure the woman's upper torso. Also, current bra fitting techniques do not measure each individual breast in a non-sagging position so that a proper fitting bra that supports each breast in a non-sagging position may be selected.

Bra fitting is generally regarded as an art and not science. The general guides for fitting a bra typically include the following steps: (1) determining the desired bra style and fabric, (2) determining the proper band size; (3) determining the proper center panel size, (4) determining the proper cup size, and (5) determining the proper strap size.

Normally, the size of each breast changes during pregnancy and breastfeeding. They typically revert to their approximate previous size after pregnancy.

### SUMMARY OF THE INVENTION

Disclosed herein is a novel upper torso and breast measurement device and bra fitting system design to address the above stated problems. The system uses the act of submerging the upper torso of a woman in a body of water to lift and re-configure the woman's breast in their natural state. By submerging each breast in a body of water, the buoyancy of each breast overcomes the gravitation forces and causes sagging. The breasts are allowed to lift and re-conform to its natural shape. A digital scanning machine is then use to measure the width and girth of the upper torso and submerged breast's exact size, volume and relative position. The data is used to select and construct a properly fitting bra which holds and supports each breast in her natural position.

In the preferred embodiment, the system includes a transparent tank filled with water in which a woman sits or stands in a fixed position. A scanning machine then moves around (or up and down) the tank and is continuously aimed at the woman's upper torso and breasts so that a 3D image of the upper torso and each breast is obtained. The exact size and shape of the upper torso and the size, volume and location of each breast is then determined. A digital file is then created and stored that is then used later to select and construct a proper individual breast fitted cup symmetrical bra.

### DESCRIPTION OF THE DRAWINGS

FIG. 1 is a side elevational view of a woman sitting in a transparent tank filled with sufficient amount of water so that her breasts are submerged with a 3D scanning machine that moves around the tank and takes 3D photographs of the woman's breasts.

FIG. 2 is a top plan view of the transparent tank filled with water showing the movement of the 3D scanning machine around the tank.

FIG. 3 is a block diagram depicting the steps carried out when scanning a woman's breast with the device shown in FIGS. 1 and 2.

FIG. 4 is a side elevational view of a woman's breast showing how a sagging breast submerged in water changes its shape and size.

### DESCRIPTION OF THE PREFERRED EMBODIMENT(S)

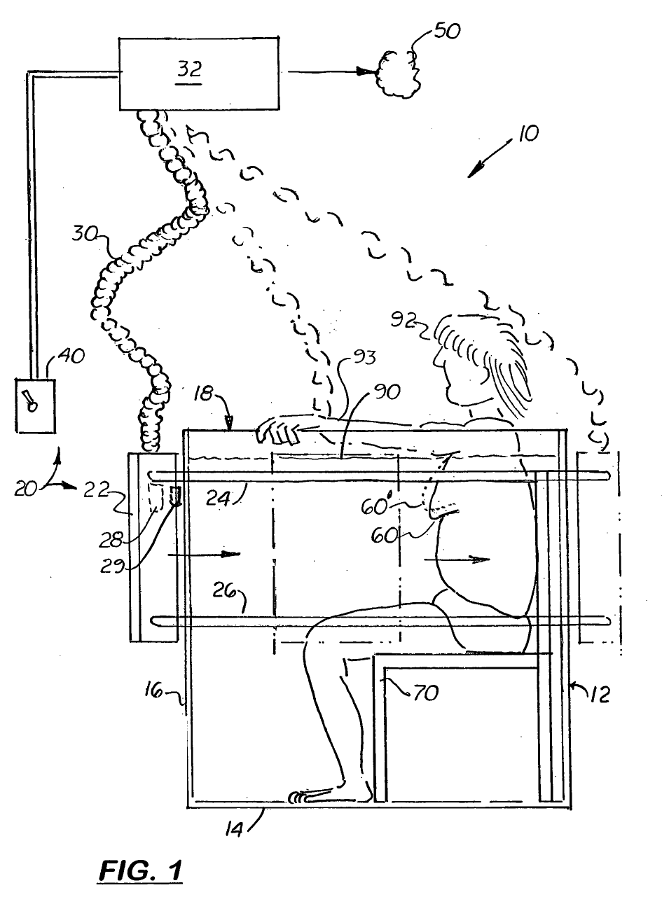

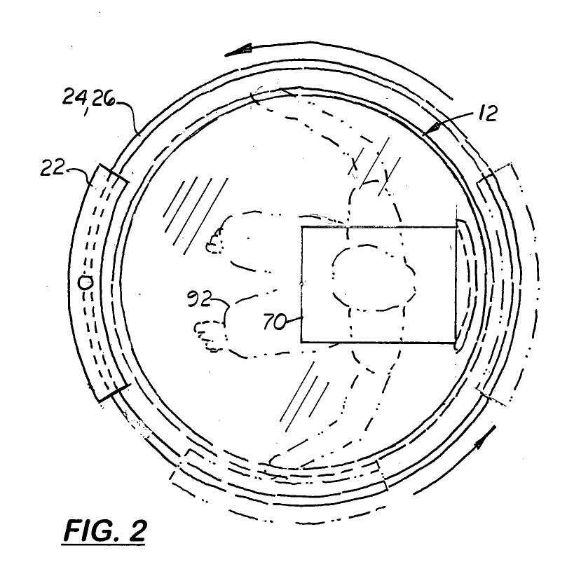

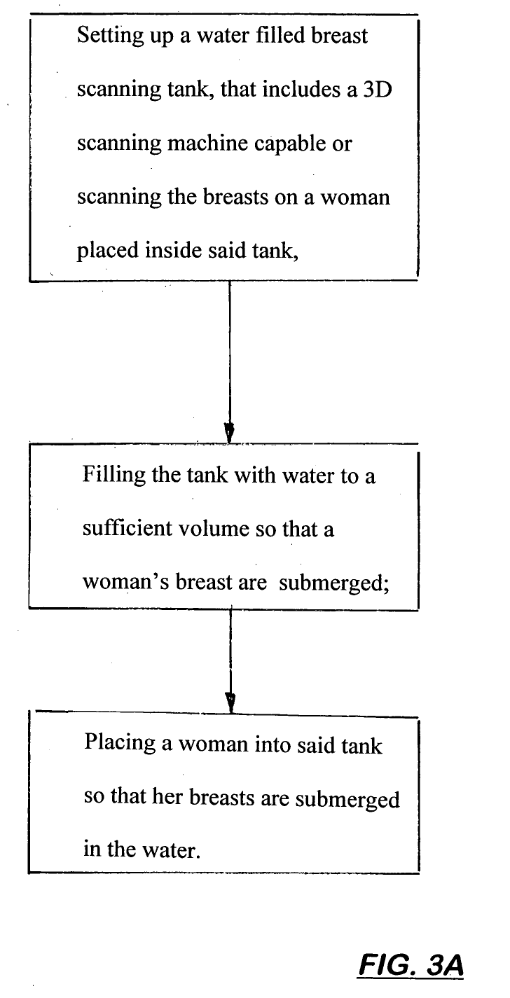

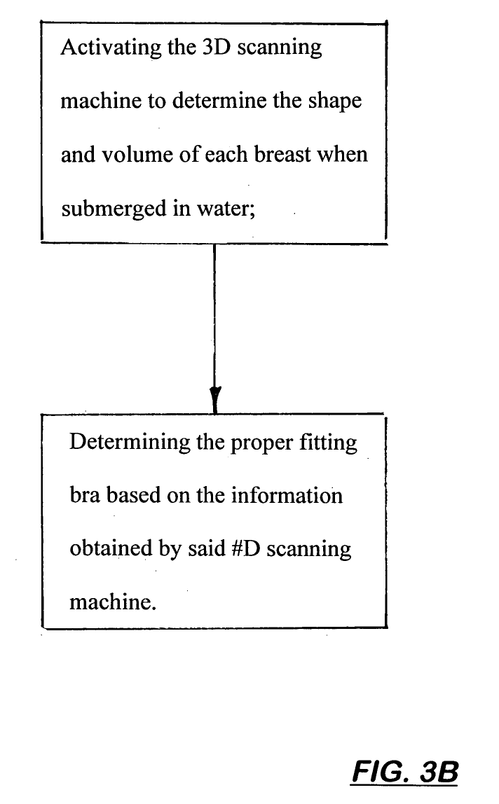

Referring to FIGS. 1 and 2 there is shown a device 10 used to accurately measure the size and shape of a woman's breast for proper fitting a bra. The device 10 includes a large tank 12 capable of holding a large body of water 90. In the embodiment shown in FIGS. 1 and 2, the tank 12 is a cylindrical with a bottom surface 14 and a continuous side wall 16. The top 18 of the tank 12 is open. The side wall 16 of the tank 12 is made of rigid, transparent material, such as glass or plastic that enables scan images to be taken by a scanner box 22 that moves over the exterior of the tank 12. In the preferred embodiment, the tank 12 is filled with salt water with the water's salinity being adjusted according to the individual's body mass index (BMI).

Surrounding the tank 12 is a scanner assembly 20 designed to take a scanned image of a woman's breast while sitting in the tank 12. In the embodiment shown herein, the scanner assembly 20 is a 3D scanner assembly that includes two rigid, horizontally aligned circular side rails 24, 26 and a moving scanner box 22. The scanner box 22 includes a motor mechanism 28 that causes the scanner box 22 to smoothly and slowly move over the two side rails 24, 26 in a 270 to 360 degree circular path. Located inside the scanner box 22 is a scanning head 29. The scanner box 22 is tethered to a cable 30 that extends downward from a control box 32 located along the ceiling directly above the tank 12. An operation control switch 40 is mounted nearby and connected to the control box 32 to selectively control operation of the motor 28 and scanner head 29 inside the scanner box 22. When a scan is completed, a digital file 50 containing the scanned image of the woman's upper torso and the two breasts, which are lifted when submerged in the water 90. FIG. 1 depicts the original position of the breast 60 before being submerged and the breast 60′ after being submerged. The submerged breasts 60′ are then used to select and construct a proper fitting bra with the correct cup, center panel, band, and strap size.

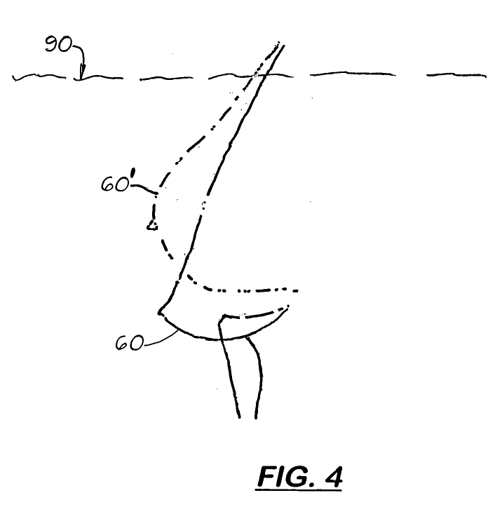

Using the above device 10, a novel method for accurately measuring a woman's breasts 60 for determining a proper bra fit is provided that submerges the upper torso 94 of a woman 92 in a large tank 12 filled with water 90. By submerging the breasts 60′ in a body of water 90, the buoyancy of the breasts 60′ allows them to lift and re-conform to their un-stretched natural shape as shown in FIG. 4. The image file 50 showing the shape and size of the re-shaped, submerged breast 60′ is then used to select a properly fitting bra 100 which fits comfortably around the upper torso and holds and supports the woman's breast 60 in the natural position when submerged.

In the preferred embodiment, the woman 92 sits on a chair 70 in a stationary position inside the tank 12. The woman raises her arms so that they may rest on the top edge of the tank 12. The tank 12 is then filled with water 90. After approximately five minutes, the scanner box 22 is then activated to move in a circular path approximately 270 to 360 degrees around the tank 12 and continuously aimed at the woman's upper torso and her two breasts 60′. The scanned images are then saved in a digital file 50 which may be given to the woman or delivered to a bra or lingerie store.

An example of a 3D scanner that may be easily modified and used in the above described system and method is available from Cyperware System located in Monterey, Calif. (USA).

In compliance with the statute, the invention described herein has been described in language more or less specific as to structural features. It should be understood however, that the invention is not limited to the specific features shown, since the means and construction shown, is comprised only of the preferred embodiments for putting the invention into effect. The invention is therefore claimed in any of its forms or modifications within the legitimate and valid scope of the amended claims, appropriately interpreted in accordance with the doctrine of equivalents.

I claim:

1. A breast measurement device, comprising:
a. a tank capable of holding a woman with exposed breasts and a sufficient amount of water so that said breasts are submerged in said water, said tank being made of transparent material;
b. a 3D scanning box located along the outer surface of said tank and aimed at the breasts of the woman located inside the tank said 3D scanning box able to take scanned images of the breast of the woman and saving the 3D images as a digit file; and,
c. a means for moving said scanning box around said tank in order to obtain a 3D scanned image of the breasts submerged in water.

2. A method of accurately measuring a woman's breasts and upper torso for determining a proper bra fit, comprising the following steps:
a. selecting a 3D submerged breast imaging apparatus that includes a tank filled with water and a 3D scanning box to rotate around said tank and take 3D images of the breast of a woman located inside said tank;
b. submerging the upper torso of a woman in the tank filled with water that allows the buoyancy of the breasts when submerged to lift and re-conform to their natural shape;
c. activating a scanning machine aimed at the breasts to measure their exact size and volume; and,
d. selecting a bra using the exact measurement information from said scanning machine designed to hold each breast in a lifted, re-conformed configuration.
